(19) United States
(12) Patent Application Publication
(10) Pub. No.: US 2013/0165832 A1
(43) Pub. Date: Jun. 27, 2013

(54) REHABILITATION GLOVE

(71) Applicant: Tzyy Jen KUO, Taichung City (TW)

(72) Inventor: Tzyy Jen KUO, Taichung City (TW)

(21) Appl. No.: 13/337,849

(22) Filed: Dec. 27, 2011

(51) Int. Cl. A61F 5/00

(52) U.S. Cl. 602/21

(57) ABSTRACT

A rehabilitation glove includes flexible glove-shaped inner and outer layers and at least five rigid support strips. The inner and outer layers are stitched at a portion between thumb and index finger, a portion below thumb, and a portion below little finger in such a manner that reduced entry openings are formed to thereby define channels, into which the rigid support strips are insertable to support and be substantially in registration with fingers. An elongate fabric band is attached to the rehabilitation glove for selectively wrapping around an outer circumference of the rehabilitation glove. The elongate fabric band has a free end to which a fastening structure in attached, whereby the elongate fabric band can wrap around the outer circumference of the rehabilitation glove and the fastening structure can then fix the elongate fabric band and the rehabilitation glove, the rigid support strips, and palm together.

### BACKGROUND OF THE INVENTION

1. Field of the Invention

The present invention relates to the field of rehabilitation tools, and in particular to a rehabilitation glove comprising an inner layer that receives a hand therein and an outer layer between which channels are formed to receive and set rigid support strips at locations corresponding to digits and fixed in position by being wrapped by an elongate fabric band in order to prevent the occurrence of atrophy and curling of finger muscles.

2. The Related Arts

The capability of muscles is generally following the theory of “use and disuse”. In other words, the more frequently the muscles exercise, the stronger the muscles will be, otherwise the muscles get weaker capability. An important issue is that it is generally not possible to identify any deterioration of muscles at the beginning of deterioration from the outside appearance of the muscles. The only way to maintain the power and strength of muscles is consistent exercising of muscles. For those lying long term on sickbeds, the potential risk of no intensive use of muscles would gradually lead to muscle atrophy, deterioration of muscle ability, deformation of joints, and poor blood circulation. Thus, it has always been a task to protect a long-lying patient against atrophy and curling of finger muscles and deformation of joints and/or to improve blood circulation. A generally adopted way of handling this issue is consistently massaging the patient's hands. This is generally a labor- and time-consuming job and doing consistent massage may easily cause tiredness of the attendant. Thus, improvements in this respect are desired.

Further, when fingers get hurt, the therapeutic process often needs to place a plate to fix the fingers. The conventional way of fixing fingers is generally hard to practice and it is bad looking by tying a plate outside the fingers.

### SUMMARY OF THE INVENTION

An objective of the present invention is to provide a rehabilitation glove, which comprises a flexible glove-shaped inner layer, a flexible glove-shaped outer layer, and at least five rigid support strips. The inner layer and the outer layer are stitched at a portion between thumb and index finger, a portion below thumb, and a portion below little finger in such a manner that reduced entry openings are formed to thereby define channels, so that when the inner layer receives a hand of a user therein, the rigid support strips are then inserted into the channels between the inner layer and the outer layer to be substantially in registration with fingers. An elongate fabric band is attached to the rehabilitation glove for selectively wrapping around an outer circumference of the rehabilitation glove. The elongate fabric band has a free end to which a fastening structure in attached, whereby the elongate fabric band can wrap around the outer circumference of the rehabilitation glove and the fastening structure can then fix the elongate fabric band and the rehabilitation glove, the rigid support strips, and palm together in order to prevent atrophy and curling of finger muscle.

Another objective of the present invention is to provide a rehabilitation glove that is applicable to damaged or bone-fractured fingers for correcting so as to realize fast fixing and provide a better looking.

A further objective of the present invention is to provide a rehabilitation glove that comprises rigid support strips that are perforated to form a plurality of through holes for improving air ventilation of the rigid support strips.

### BRIEF DESCRIPTION OF THE DRAWINGS

The present invention will be apparent to those skilled in the art by reading the following description of a preferred embodiment thereof, with reference to the attached drawings, wherein:

FIG. 1 is an exploded view of a rehabilitation glove according to an embodiment of the present invention;

FIG. 2 is an assembled view of the rehabilitation glove according to the embodiment of the present invention, illustrated in a viewing through way;

FIG. 3 is a perspective view, in a cut-away form, of the rehabilitation glove according to the embodiment of the present invention;

FIG. 4 is a schematic view illustrating an elongate fabric band wrapping around the rehabilitation glove according to an embodiment of the present invention;

FIG. 5 is a schematic view illustrating the rehabilitation glove of the present invention applied to a left hand of user; and

FIG. 6 is a schematic view illustrating the rehabilitation glove of the present invention applied to a right hand of user.

### DETAILED DESCRIPTION OF THE PREFERRED EMBODIMENT

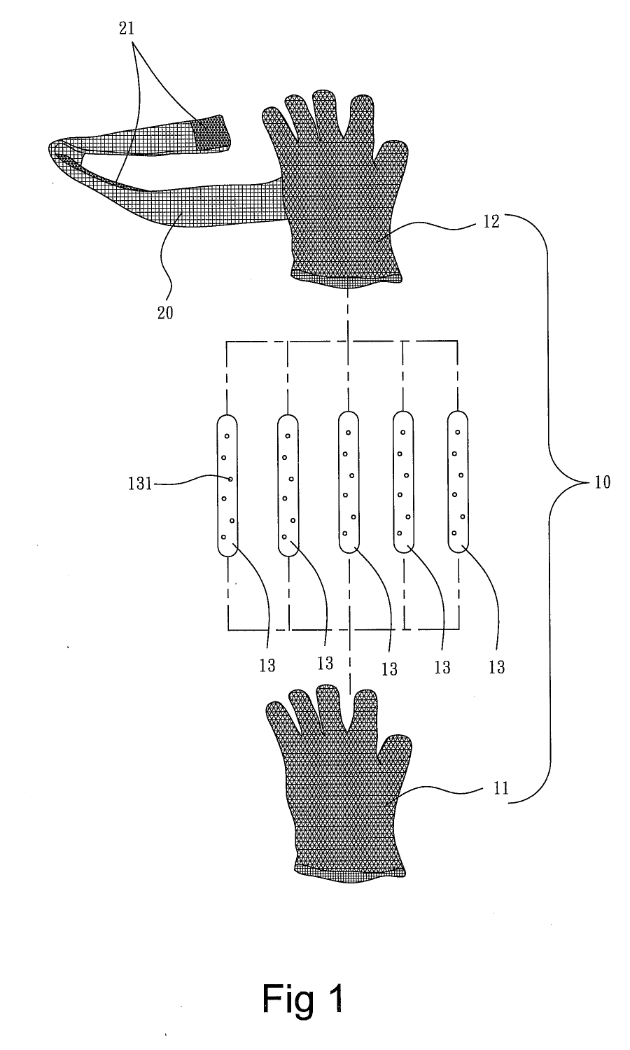

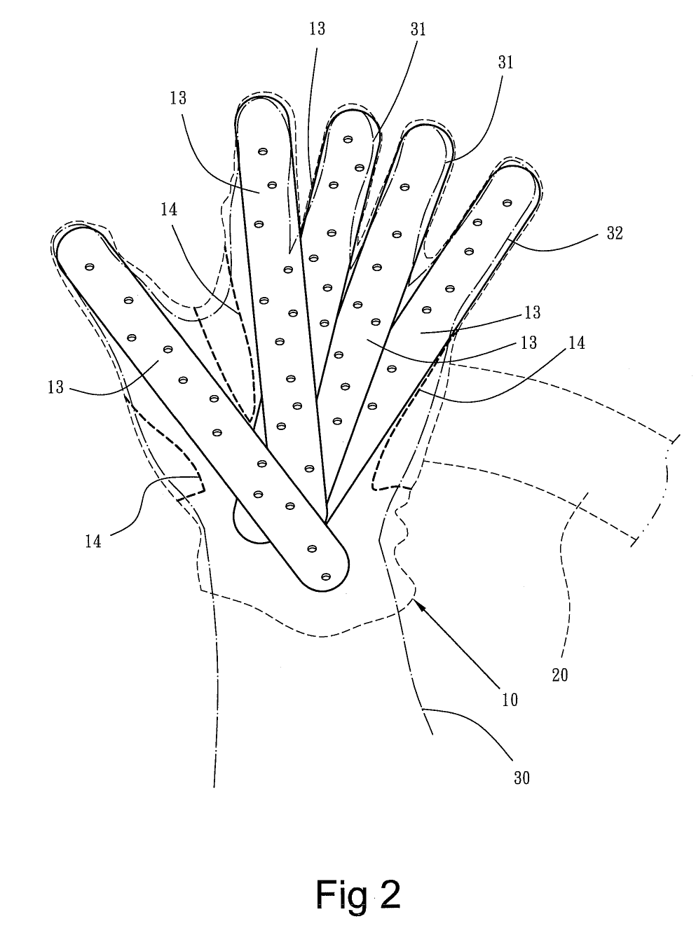

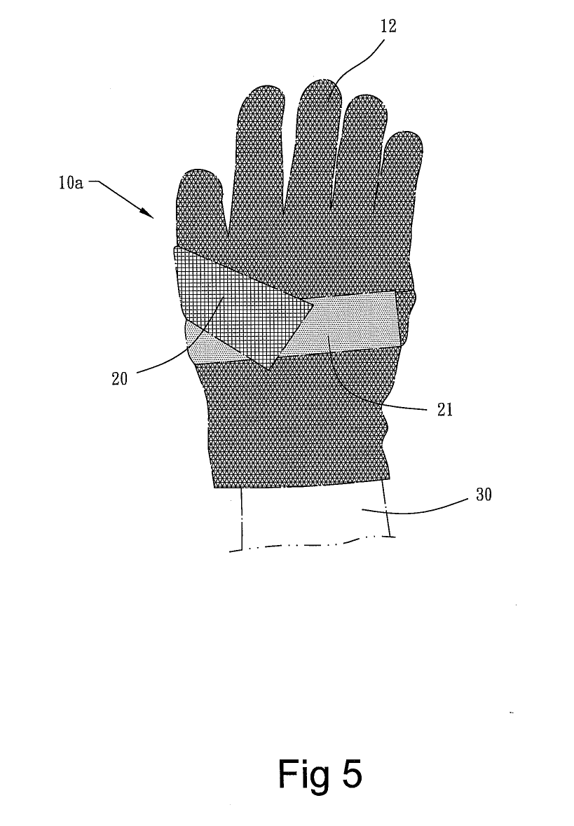

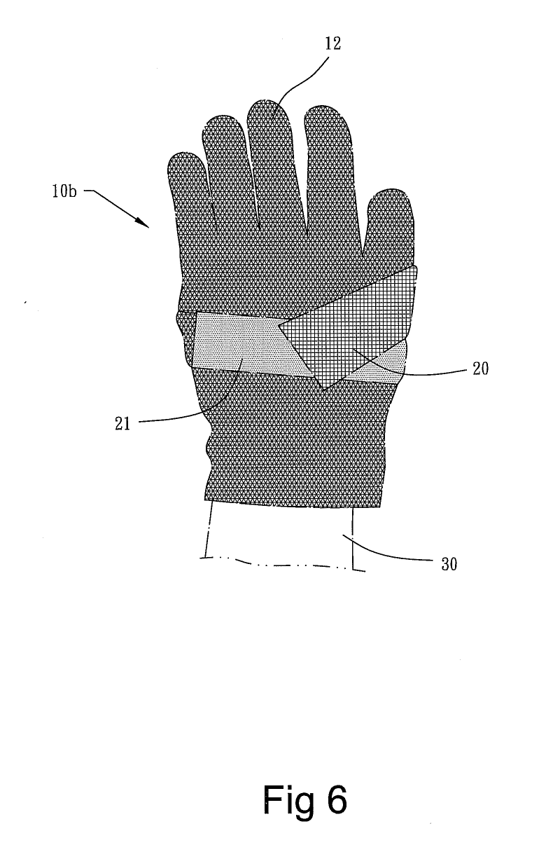

With reference to the drawings and in particular to FIGS. 1 and 2, which are respectively an exploded view and an assembled view of a rehabilitation glove according to the present invention, the rehabilitation glove of the present invention can be made a sinister or left-hand glove, such as the glove 10a shown in FIG. 5, or a dexter or right-hand glove, such as the glove 10b shown in FIG. 6. For simplicity, in the following description, these gloves will be collectively designated by reference 10.

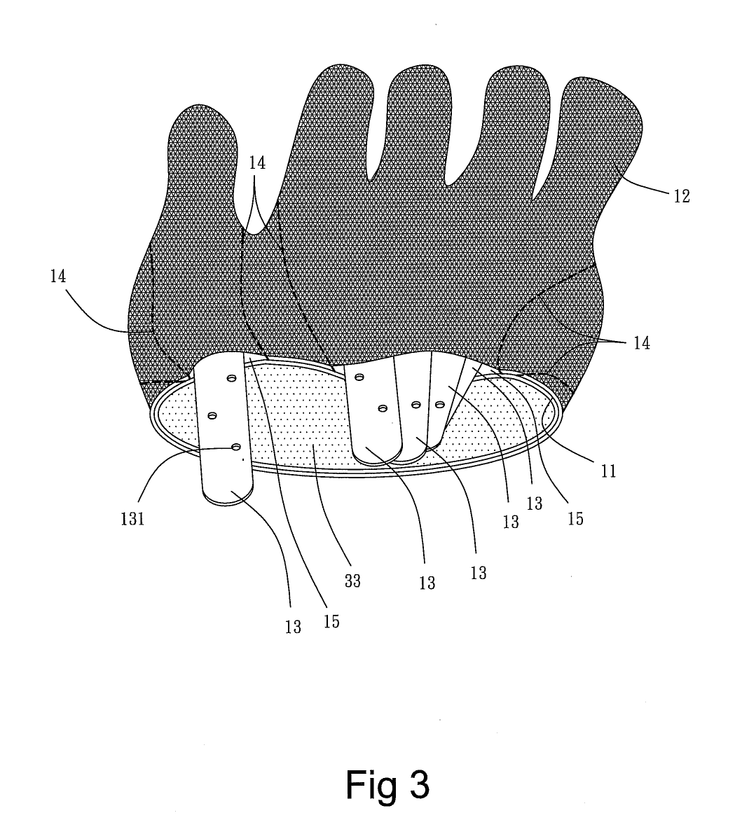

The rehabilitation glove 10 of the present invention comprises a flexible glove-shaped inner layer 11, a flexible glove-shaped outer layer 12, and five rigid support strips 13. The inner layer 11 and the outer layer 12 are fixed to each other by stitching 14 at the portion between thumb and index finger, the portion below thumb, and the portion below little finger in such a manner that reduced entry openings are formed to thereby define channels 15 (as shown in FIG. 3). With such an arrangement, after a user's hand 30 is fit into the inner layer 11, the rigid support strips 13 can be inserted into the channels 15 between the inner layer 11 and the outer layer 12 and located substantially in registration with the fingers 31. The rehabilitation glove 10 also comprises an elongate fabric band 20 that has an end attached to one side of the rehabilitation glove 10 at a location close to and below the little finger 32. The elongate fabric band 20 can be selectively used to wrap around the outer circumference of the rehabilitation glove 10. The elongate fabric band 20 has a free end to which a fastening structure 21, such as hook-and-loop fastener (which is also referred to as “Velcro”), is attached. The elongate fabric band 20 can wrap around the outer circumference of the rehabilitation glove 10 and the fastening structure 21 can then fix the elongate fabric band 20 and the rehabilitation glove 10, the rigid support strips 13 and the user's palm 33 (see FIG. 3) together in order to prevent atrophy and curling of finger muscles.

The rigid support strips 13 of the rehabilitation glove 10 may be perforated and form a plurality of through holes 131 to improve air ventilation of the rigid support strips 13.

The rehabilitation glove 10 may be applicable to fixing damaged and bone-fractured fingers. The damaged hand is first fit into the rehabilitation glove 10 and the rigid support strips 13 are inserted into the channels 15 between the inner layer 11 and the outer layer 12 to be substantially in registration with the fingers. The elongate fabric band 20 is then wrapped around the outer circumference of the rehabilitation glove 10. The elongate fabric band 20 may be wrapped several turns around the outer circumference of the rehabilitation glove 10 and then the fastening structure 21 attached to the free end of the elongate fabric band 20 is applied to fix the elongate fabric band 20, the rigid support strips 13, and the user's palm. The use is easy and the outside looking is good.

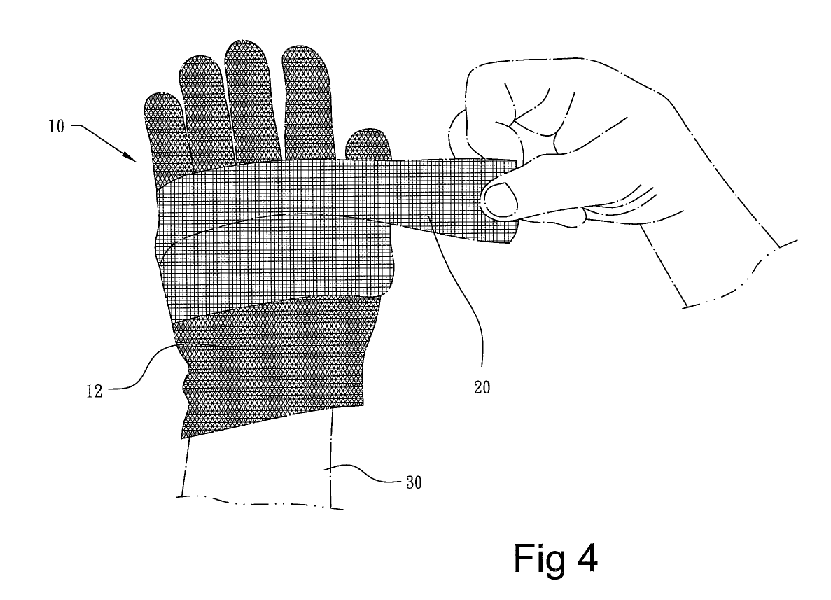

Referring to FIGS. 3-6, the rehabilitation glove 10 of the present invention can be used as a rehabilitation tool, which can be a sinister rehabilitation glove 10a (see FIG. 5), or a dexter rehabilitation glove 10b (see FIG. 6), or even a pair of rehabilitation gloves 10 and comprises at least five rigid support strips 13.

The rehabilitation glove 10 is of a double-layered structure, including an inner layer 11 and an outer layer 12. Finger 31 of a person to take rehabilitation are fit into the inner layer 11 of the rehabilitation glove 10 and the rigid support strips 13 are inserted between the inner layer 10 and the outer layer 12. Stitching is applied to the inner layer and the outer layer 12 to form channels 15 between the two layers for receiving and retaining the rigid support strips 13 in position without undesired shifting or sliding. An elongate fabric band 20 is attached, with an end thereof, to one side of the rehabilitation glove 10 at a location close to and below the little finger 32. The elongate fabric band 20 can wrap the outer circumference of the rehabilitation glove 10 by one or more turns. A fastening structure 21 of which an example is the hook-and-loop fastener is at attached to a free end of the elongate fabric band 20 so that after the elongate fabric band 20 is wrapped around the outer circumference of the rehabilitation glove 10, the fastening structure 21 may be applied to fix the elongate fabric band 20.

The operation of the present invention is described as follows. Fingers of a person to take rehabilitation are first fit into the inner layer 11 of the rehabilitation glove 10 and then the five rigid support strips 13 are inserted between the inner layer 11 and the outer layer 12. The elongate fabric band 20 is then wrapped around the palm and is fixed to the rehabilitation glove 10 by the fastening structure 21. This completes the operation. Both left and right hands can be handled in this way.

The present invention uses the flexibility of the rehabilitation glove 10 to comply with the curling fingers in order to allow fingers to easily fit into the rehabilitation glove 10. This, when used in combination with the supporting forces provided by the rigid support strips 13, may effectively extend the curling fingers to achieve the purposes of preventing curling and deformation of fingers and maintain proper angles of the finger joints to thereby reduce the extents of atrophy and deformation of joints and muscles of finger. The elongate fabric band 20 that is attached to the side of the glove is provided for improving tightness between the palm and the rigid support strips 13, making the palm tightly against the rigid support strips 13 to thereby improve the result of expanding fingers. The rehabilitation glove 10 can be used to fix, maintain, control, and correct finger muscles and to prevent deformation of finger joints and atrophy and deterioration of finger muscles, and is helpful in maintaining blood circulation of finger and the perception of action.

Although the present invention has been described with reference to the preferred embodiment thereof, it is apparent to those skilled in the art that a variety of modifications and changes may be made without departing from the scope of the present invention which is intended to be defined by the appended claims.

What is claimed is:

1. A rehabilitation glove, comprising:
a flexible glove-shaped inner layer, a flexible glove-shaped outer layer, and at least five rigid support strips, the inner layer and the outer layer being subjected to stitching at a portion between thumb and index finger, a portion below thumb, and a portion below little finger in such a manner that reduced entry openings are formed to thereby define channels, the inner layer being adapted to receive a hand of a user therein, the rigid support strips being insertable into the channels between the inner layer and the outer layer to be substantially in registration with fingers, an elongate fabric band being attached to the rehabilitation glove for selectively wrapping around an outer circumference of the rehabilitation glove, the elongate fabric band having a free end to which a fastening structure in attached, whereby the elongate fabric band can wrap around the outer circumference of the rehabilitation glove and the fastening structure can then fix the elongate fabric band and the rehabilitation glove, the rigid support strips, and palm together in order to prevent atrophy and curling of finger muscles.

2. The rehabilitation glove as claimed in claim 1, wherein the rigid support strips are perforated and form a plurality of through holes to improve air ventilation of the rigid support strips.

3. The rehabilitation glove as claimed in claim 1, wherein the rehabilitation glove is adapted to fix damaged and bone-fractured fingers, whereby the damaged hand is first fit into the rehabilitation glove and the rigid support strips are inserted into the channels between the inner layer and the outer layer to be substantially in registration with the fingers, the elongate fabric band being then wrapped around the outer circumference of the rehabilitation glove, so that after the elongate fabric band is wrapped around the outer circumference of the rehabilitation glove, the fastening structure attached to the free end of the elongate fabric band is applied to fix the elongate fabric band, the rigid support strips, and the palm to thereby achieve easy use and good looking.
